(19) United States
(12) Patent Application Publication
(10) Pub. No.: US 2025/0207206 A1
(43) Pub. Date: Jun. 26, 2025

(54) MODIFICATION-DEPENDENT ENRICHMENT OF DNA BY GENOME OF ORIGIN

(71) Applicants: The Board of Trustees of the Leland Stanford Junior University, Stanford, CA (US); The United States of America, as represented by the Secretary, Department of Health and Human Servic, Bethesda, MD (US)

(72) Inventors: Syed Usman Enam, Redwood City, CA (US); Andrew Z. Fire, Redwood City, CA (US); David Lipman, Redwood City, CA (US); Susan Leonard, Redwood City, CA (US); Joshua L. Cherry, Redwood City, CA (US); Ivan Zheludev, Stanford, CA (US)

(21) Appl. No.: 18/847,077

(22) PCT Filed: Mar. 30, 2023
(86) PCT No.: PCT/US2023/016926

§ 371 Date: Sep. 13, 2024

Related U.S. Application Data
(60) Provisional application No. 63/326,073, filed on Mar. 31, 2022.

(51) Int. Cl. C12Q 1/689; C12N 15/10; C12Q 1/44; C12Q 1/6806; C12Q 1/683; C12Q 1/6869

(52) U.S. Cl. CPC C12Q 1/689; C12N 15/1003; C12N 15/1093; C12Q 1/44; C12Q 1/6806; C12Q 1/683; C12Q 1/6869; C12Q 2600/148; G01N 2333/922

(57) ABSTRACT

Compositions and methods are provided to enrich for DNA corresponding to a genome of interest, e.g. by species, clade, or strain of origin, from a mixed population of nucleic acid sequences. The methods may further comprise identification of the genomic sequences of interest, e.g. identifying the species, clade, strain, etc. of origin.

### CROSS REFERENCE TO OTHER APPLICATIONS

This application claims the benefit of U.S. Provisional Application No. 63/326,073, filed Mar. 31, 2022, the contents of which are hereby incorporated by reference in its entirety.

### GOVERNMENT RIGHTS

This invention was made with Government support under contracts GM130366 and HG000044 awarded by the National Institutes of Health. The Government has certain rights in the invention.

### BACKGROUND

Foodborne pathogen outbreaks can be a major public health and agro-economic burden. According to the World Health Organization, one in ten people are victim to foodborne illnesses every year. When such outbreaks occur, food and agricultural safety organizations are tasked with determining the responsible contaminated food, the pathogen causing the illness and the source of this pathogen so that required measures can be taken to remove implicated food products from commerce and perform remediation steps to prevent further illnesses.

Specific strains of enterobacteria are amongst the common pathogens linked to foodborne illnesses (e.g. Salmonella enterica, Shiga toxin-producing Escherichia coli). In an outbreak setting, pathogen detection and identification are often achieved through serotype testing, DNA marker amplification, or targeted sequencing of genomic loci, but these methods sometimes provide insufficient information to trace the organism back to its source. Thus, strain and sub-strain level information encoded by single nucleotide polymorphisms (SNPs) can have great value in tracking and matching a pathogen to its environmental source. Whole genome sequencing (WGS), unlike targeted methods, provides this information and is used at various checkpoints between the farm and consumer to monitor and control contamination in produce.

WGS of an outbreak pathogen is obtained through either a culture-dependent or a culture-independent approach, the first of which can add many days and substantial cost to an investigation depending on how simple it is to isolate the pathogen in question and the pathogen load in the sample. In a culture-independent approach, shotgun sequencing is performed on the sample (produce, food, soil, plant) potentially containing the pathogen and assembly of the pathogen's DNA allows for rapid strain-level identification without the need for isolation. While shotgun WGS is recognized as a powerful tool for this application, it comes with the limitation that a sample needs to contain a sufficient load of the pathogen for high enough coverage of the genome to SNP map the genome. Often these samples, instead, contain irrelevant prokaryotic and eukaryotic DNA that far exceeds that of the relevant strain. This leads to low-coverage assemblies of the pathogen or increased cost due to excess sequencing of a sample.

Methods exist to deplete “host” or eukaryotic DNA and enrich for prokaryotic DNA. Some commercial kits selectively lyse eukaryotic (mostly mammalian) cells and degrade accessible DNA through enzymatic or chemical means before purifying DNA from the remaining cells. While these offer substantial depletion of eukaryotic DNA, they may fall short in several ways: 1) unable to digest (and therefore deplete) cells with robust cell walls such as fungi, 2) unable to enrich for prokaryotic DNA post-DNA extraction, 3) unable to preserve prokaryotic cell-free DNA in a sample and 4) unable to deplete irrelevant prokaryotic DNA.

Other methods deplete eukaryotic DNA post-DNA extraction by binding and sequestering this DNA due to the differential presence of methylation patterns between prokaryotes and eukaryotes. One commercial kit takes advantage of the increased presence of CpG (C5 position of cytosine) methylation in eukaryotes and uses an engineered methyl-CpG binding domain conjugated to an antibody to remove CpG methylated DNA. However, studies frequently report weak enrichment through this method likely due to the presence of large stretches of eukaryotic DNA that are not methylated. Additionally, many eukaryotes, such as Caenorhabditis elegans, exhibit predominantly unmodified DNA. A different protocol makes use of methylation-sensitive restriction enzyme Hpall in non-catalytic conditions to bind and enrich for non-CpG methylated (and therefore prokaryotic) DNA, or applies this paradigm to a different restriction enzyme, Dpnl which selectively targets methylated 5′-GATC-3′ motifs (N6 position of adenine). These motifs are methylated by Dam, a type-II methyltransferase widespread in Gammaproteobacteria (of which E. coli, S. enterica and Vibrio cholerae are members) and not found in eukaryotes. While offering substantial enrichment, these protocols are time- and cost-prohibitive as they involve using 1:1 stoichiometric amounts of enzyme to the to-be-enriched substrate DNA, modification of the enzyme by biotinylation and a final dialysis step.

For many purposes, there is a need to enrich a mixed population of DNA from several or many species, obtaining a subset of the DNA from one or more species of interest. Such purposes may include source identification of contaminants in food, environmental samples, biological (including clinical) samples, and the like.

### SUMMARY

Compositions and methods are provided to enrich for DNA corresponding to a genome of interest, e.g. by species, clade, or strain of origin, from a mixed DNA population. The methods may further comprise a step of identification of the genomic sequences of interest, e.g. identifying the species, clade, strain, etc. of origin.

In some embodiments, the methods provide for enrichment of prokaryotic genomic sequences from eukaryotic genomic sequences. In some embodiments, prokaryotic genomic sequences comprise pathogen DNA, including without limitation Enterobacteriaceae DNA. Mixed populations of nucleic acid sequences may include, without limitation, samples suspected of containing prokaryotic DNA, e.g. Enterobacteriaceae DNA, where the proportion of prokaryotic DNA in the population may be less than about 50%, less than about 33%, less than about 25%, less than about 10%, less than about 5%, less than about 1%, or less. In some embodiments, the genome of interest corresponds to less than about 25% of the total nucleic acid in the population, less than about 10%, less than about 5%, less than about 1%, less than about 0.5%, less than about 0.1%, less than about 0.05%, less than about 0.01% of the total nucleic acid in the population.

The methods of the disclosure take advantage of DNA modifications, including, without limitation, modifications such as methylation, glucosylation, etc. in the genome of interest. In some embodiments the modifications are present in specific sites, e.g. when associated with Dam methylation, Dcm methylation, Campylobacter transformation system methyltransferase (ctsM), etc. In some embodiments modified bases are present throughout a genome, e.g. modified bases found in virus genomes, etc. The presence of these modifications can make the DNA resistant to enzymatic digestion.

In some embodiments, a method for genome enrichment comprises selective endonuclease digestion of a nucleic acid sample of interest, where the sample is suspected of containing DNA that is modified such that the modified DNA, or unmodified DNA, is resistant to enzymatic endonuclease digestion. In some embodiments the DNA modification is one or both of Dam and Dom methylation. However, one of skill in the art will understand that many restriction/modification systems are found in microbes and can be used for this purpose. In some embodiments the modification is both Dam and Dom methylation. The nucleic acid sample of interest is digested with one or a cocktail of enzymes, e.g. two, three or more enzymes, which enzymes selectively digest either the unmodified DNA or the modified DNA. In some embodiments a cocktail of enzymes specific for one or more different recognition sites is used, for example, where at least one enzyme is blocked by Dcm modification and at least one enzyme is blocked by Dam methylation. Enzymes of interest for this purpose include, without limitation, PspGI, EcoRII, Mbol, and isoschizomers thereof that are similarly blocked by Dcm or Dam methylation, and enzymes such as Dpnl that are dependent on specific methylation.

Following the step of selective endonuclease digestion, the population of DNA is manipulated to preferentially retain longer, uncleaved fragments, for example by size selection. In some embodiments, size selection is performed by exonuclease degradation of the population of endonuclease cleaved DNA. In some embodiments, the exonuclease is a distributive exonuclease. In some embodiments, the exonuclease is distributive T5 exonuclease. The exonuclease treatment selectively eliminates short fragments from the endonuclease treatment, leaving longer, uncleaved DNA fragments. Following exonuclease treatment, longer undigested DNA that is resistant to endonuclease cleavage is, on average, usually greater than about 5 Kb in length, greater than about 10 Kb, greater than about 15 Kb, greater than about 20 Kb, greater than about 25 Kb, or more.

In other embodiments, size selection is performed by gel electrophoresis, where the gel is appropriate to separate uncleaved DNA that is, on average, usually greater than about 5 Kb in length, greater than about 10 Kb, greater than about 15 Kb, greater than about 20 Kb, greater than about 25 Kb from smaller cleaved DNA. Following electrophoresis, the DNA fragments of interest are excised and eluted from the gel.

The longer, separated DNA fragments, corresponding to the genome of interest, can be used for amplification, library preparation, direct sequencing, and the like; particularly to identify the species, clade, strain, etc. of origin of the genome of interest. The level of enrichment is usually at least about 10-fold relative to the starting population, at least about 15-fold, at least about 20-fold, at least about 25-fold, or more.

In an embodiment, a method is provided for characterizing specific types of microbial genomes in a sample, the method comprising obtaining nucleic acids from a sample of interest, where the sample potentially comprises a mixture of microbial DNAs with or without nonmicrobial DNA; treating the nucleic acid sample with a cocktail of enzymes specific for at least one, or at least two, different recognition sites, where the enzymes are blocked by methylation (or lack of) at said recognition sites; treating the endonuclease digested DNA with a distributive DNA exonuclease for a period of time sufficient to selectively eliminate shorter, endonuclease cleaved fragments; and identifying the remaining DNA by species, clade, strain, etc. of origin, including identification of genomic sequences of interest. In some such embodiments the microbial DNA includes microbial pathogen DNA, e.g. DNA from a pathogenic Enterobacteriaceae. In some embodiments the sample is a biological sample, e.g. a clinical sample. In some embodiments the sample is a food sample. In some embodiments the sample is a pharmaceutical sample. In some embodiments the sample is an environmental sample.

In some embodiments, kits are provided for practice of the methods of the disclosure. Kits may comprise, for example, one or cocktail of endonucleases, for example a cocktail of enzymes specific for one or more different recognition sites, including without limitation where at least one enzyme is blocked by Dcm modification and at least one enzyme is blocked by Dam methylation; and a distributive enonuclease. Kits may further comprise buffers and reagents suitable for carrying out digestions; reagents for sequencing, instructions for use; and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: Schematic of the pipeline for endonuclease and exonuclease-based enrichment of methylated DNA. A metagenomic sample containing DNA that is and is not Dam and Dem methylated is treated with methylation sensitive enzymes. The unmethylated DNA is digested to short fragments while the methylated DNA remains long and intact. Size selection for longer fragments is performed with either electrophoretic separation or a distributive exonuclease (which preferentially degrades short fragments). The enriched sample is then sequenced.

FIGS. 2A-2D: Methylation sensitive endonucleases and T5 exonuclease enrich for E. coli DNA in an E. coli and C. elegans DNA mixture (A) Gel showing the susceptibility of either E. coli DNA or C. elegans DNA to PspGI, Mbol and EcoRII separately and all together. The genomic high molecular weight C. elegans band disappears when the endonuclease is applied. (B) Gel showing timepoints of T5 exonuclease treatment when applied to a 1:3 mixture of E. coli to C. elegans DNA treated with the corresponding endonucleases. Notice the disappearance of the low molecular weight smear (C. elegans DNA) with longer T5 exonuclease incubation. (C) Paired end sequencing data from untreated and treated samples. In blue are the proportion of reads in that sample that map to the E. coli genome and in yellow are the proportion that map to the C. elegans genome. Any reads that do not map to either or have chimeric paired reads are colored grey. The C. elegans only sample contains a certain amount of E. coli DNA likely due to the fact that the worms are fed E. coli. Shown below is the relative enrichment of E. coli DNA calculated as the ratio of the number of E. coli reads to C. elegans reads divided by the ratio in the untreated control. (D) For each T5 exonuclease time point, all C. elegans reads that remained were mapped to the length of the theoretical fragment size that they would be found in an in silico digestion of the C. elegans genome. A cumulative density plot of these fragments is shown to ascertain whether remaining C. elegans reads originate from long fragments or short fragments.

FIGS. 3A-3B: Methylation sensitive endonucleases and various size-selection approaches enrich for E. coli and S. enterica DNA in the Zymo mix. (A) Paired end sequencing data from untreated, endonuclease-only treated and endonuclease as well as T5 exonuclease treated DNA. In blue are reads that map to genomes that are Dam and Dcm methylated. In yellow are reads that map to genomes that are not Dam and Dcm methylated. Mean proportions of two biological replicates were plotted. Relative enrichment of E. coli and S. enterica shown below were calculated from the mean proportions. The raw enrichment values for each replicate are as follows (1.0, 0.87, 9.72) and (1.0, 0.76, 12.74) from left to right. (B) Paired end sequencing data from untreated and endonuclease treated DNA size-selected through gel electrophoresis (FIG. 8). Mean proportions of two biological duplicates were plotted. Relative enrichments of E. coli and S. enterica shown below were calculated from mean proportions. The raw enrichment values for both duplicates were (1.0, 141.8) from left to right.

FIGS. 4A-4B 4: Dynamic range of enrichment on various amounts and ratios of methylated DNA. (A) Paired end sequencing data from either half, one-tenth or one-hundredth the amount of Zymo mix DNA used in the standard protocol following otherwise the same enzyme concentrations. In blue are reads that map to genomes that are Dam and Dcm methylated. In yellow are reads that map to genomes that are not Dam and Dcm methylated. Relative enrichment is shown below. (B) Paired end sequencing data from Zymo mix DNA mixed with S. cerevisiae DNA in 1:1, 1:9 and 1:99 ratios with total amount remaining 75 ng. Mean proportions of two biological duplicates were plotted for (B). Relative enrichment of E. coli and S. enterica shown below were calculated from the mean proportions. The raw enrichment values for each replicate are as follows (1.0, 62.9, 1.0, 45.0, 1.0, 28.6) and (1.0, 81.8, 1.0, 65.1, 1.0, 35.8) from left to right. In red are reads that map to T4 phage. T4 phage DNA sequences represent a population of DNA fortuitously included with the yeast DNA material used in these assays. Notably, this DNA is enriched in parallel to the modified bacterial DNAs, a behavior that is both of interest and expected as a consequence of the known modification of T4 DNA. Thus, this population serves as a fortuitous positive control on the enrichment observed. Mean relative enrichment of T4 phage, E. coli and S. enterica, together, is (1.0, 89.5, 1.0, 193.4, 1.0, 307.8) from left to right.

FIG. 5: Dpnl and T5 exonuclease treatment enriches for DNA that is not Dam methylated. Paired end sequencing data for Zymo mix DNA treated with Dpnl which only cuts at methylated Dam sites. Relative enrichments are shown below.

FIG. 6. A 20-minute incubation of T5 exonuclease treatment enriches for E. coli DNA maximally as opposed to 5 minutes or 60 minutes. Paired end sequencing data from untreated and treated samples. In blue are the proportion of reads in that sample that map to the E. coli genome and in yellow are the proportion that map to the C. elegans genome. Any reads that do not map to either or have chimeric paired reads are colored grey. The C. elegans only sample contains a certain amount of E. coli DNA likely due to the fact that the worms are fed E. coli OP50. The difference in fold enrichment obtained here and in FIG. 2C is likely due to the presence and depletion of E. coli OP50 which is not Dcm methylated. Shown below is the relative enrichment of E. coli DNA calculated as the ratio of the number of E. coli reads to C. elegans reads divided by the ratio in the untreated control.

FIG. 7: EcoRII treatment may be omitted for endonuclease-based enrichment. Paired end sequencing data from untreated, and endonuclease as well as T5 exonuclease treated Zymo mix DNA with and without EcoRII. In blue are reads that map to genomes that are Dam and Dcm methylated. In yellow are reads that map to genomes that are not Dam and Dcm methylated.

FIG. 8: Electrophoretic Size Selection and gel extraction of endonuclease treated Zymo mix DNA. Large, undigested DNA was found above the 15 kb marker. The high molecular weight band from the untreated and endonuclease treated sample was extracted for library preparation.

FIG. 9. Read coverage of the T4 phage genome found in the yeast DNA sample. (Top) Read coverage of T4 phage found in the Yeast (w/T4) sample from FIG. 4B. (Middle) Read coverage of T4 phage found in the 1:99 Zymo mix: Yeast (w/T4) DNA untreated sample from FIG. 4B. (Bottom) Read coverage of T4 phage found in the 1:99 Zymo mix: Yeast (w/T4) DNA treated with the respective endonucleases and exonuclease from FIG. 4B. Note the enrichment and higher coverage of the T4 phage genome after REMODE.

FIG. 10. OP50 E. coli is susceptible to PspGI and EcoRII. Gel electrophoresis of OP50 DNA either untreated or treated with PspGI, Mbol and/or EcoRII. Since OP50 is not Dcm methylated, it is digested by PspGI and EcoRII.

FIG. 11. Endonuclease treatment of E. coli and C. elegans DNA preparations. Note that for C. elegans material we used a mixture of DNA (slower migrating species on gel) and RNA (faster migrating species on gel), with the results demonstrating that the presence of RNA in analyzed materials does not prevent the operation of the specific endonuclease.

FIG. 12. Exonuclease treatment of endonuclease treated DNA.

FIG. 13. Enrichment of E. coli DNA assayed via sequencing. Shown are results from sequencing DNA samples containing either E. coli DNA, C. elegans DNA or a 1:3 mixture of E. coli to C. elegans DNA with or without the endonuclease and exonuclease treatment for various lengths of time. In blue, are the proportion of reads in the sample that map to the E. coli genome and in yellow are the proportions of reads in the same sample that map to the C. elegans genome. The figure shows enrichment of E. coli DNA when treated with the corresponding endonucleases (PspGI, Mbol, EcoRII) and T5 exonuclease. Greatest enrichment of E. coli reads in this experiment was observed in the sample treated with the T5 exonuclease for 20 minutes. In this sample there is a 27.5 fold enrichment of the ratio of E. coli reads to C. elegans reads over the ratio of E. coli reads to C. elegans reads in the untreated sample.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology. A number of commercial resources are available for purchase of restriction enzymes and exonucleases, including without limitation New England Biolabs; Thermo Fisher Scientific; Promega Corporation; Sigma Aldrich; Takara Bio; etc.

Compounds and enzymes can also be synthesized by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

Sequencing assembly methods may be used, for example, to assemble multiple sequence reads into a single genome using computational approaches. Several overlapping sequence reads are pieced together to produce a single longer sequence contig. The constructed genome is aligned to a reference database for identification of the organism.

The term “isolated” refers to a molecule that is substantially free of its natural environment. For instance, an isolated protein is substantially free of cellular material or other proteins from the cell or tissue source from which it is derived. The term refers to preparations where the isolated protein is at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The term “sample” with reference to a patient encompasses environmental samples, food samples, blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc.

The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient.

Of interest are complex mixtures of cells or DNA. Samples of interest include food samples, environmental samples, e.g. hospital samples, ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Enterobacteriaceae are a family of gram-negative, rod-shaped, facultative anaerobic bacteria. Criteria for inclusion have varied, but currently a set of 50 to 200 morphologic, cultural, and biochemical features and DNA relatedness are used for classification, see for example Janda and Abbot (2021) Clinical Microbiology Reviews 34 (2) e00174-20. A key marker almost exclusively associated with this family is the enterobacterial common antigen or ECA.

Among major foodborne bacterial pathogens, the family Enterobacteriaceae is well represented by several groups, including Salmonella, Escherichia coli (O157, non-O157) including Shiga toxin-producing E. coli (STEC), Shigella, and Yersinia enterocolitica. Sources of foodborne outbreaks associated with enterobacteria include dairy, poultry, beef, pork, melons, sprouts, basil (Shigella), bagged salad (Y. enterocolitica), cookie dough and sprouted seeds (E. coli), and peanut butter and jalapeno and serrano peppers (Salmonella).

Genera in the family Enterobacteriaceae are important pathogens for three of the four major hospital acquired infections, including central line-associated bloodstream infections (CLABSI), catheter-associated urinary tract infections (CAUTI), and surgical site infections (SSI). Genera in the family include, for example, Biostraticola; Buttiauxella; Cedecea; Citrobacter; Cronobacter; Enterobacillus; Enterobacter; Escherichia; Franconibacter; Gibbsiella; Izhakiella; Klebsiella; Kluyvera; Kosakonia; Leclercia; Lelliottia; Limnobaculum; Mangrovibacter; Metakosakonia; Phytobacter; Pluralibacter; Pseudescherichia; Pseudocitrobacter; Raoultella; Rosenbergiella; Saccharobacter; Salmonella; Scandinavium; Shigella; Shimwellia; Siccibacter; Trabulsiella; and Yokenella.

Campylobacter is a genus of Gram-negative bacteria. Some Campylobacter species can infect humans, and other animals of economic interest. Among the species of Campylobacter implicated in human disease, C. jejuni, C. lari, and C. coli are common. C. jejuni is an important cause of bacterial foodborne disease. C. fetus can cause spontaneous abortions in cattle and sheep, and is an opportunistic pathogen in humans. A characteristic of most Campylobacter genomes is the presence of hypervariable regions, which can differ greatly between different strains. Campylobacter sp, e.g. C. jenuni can have methylated DNA at the motif (5′-RAATTY-3′). Apol and EcoRI can be used to selectively cleave unmodified DNA at these sites.

Restriction/Modification. Many prokaryotic microbes have developed restriction modification systems that modify DNA at a specific site, often by methylation, and cleave DNA, usually at the same site. About one quarter of known bacteria possess a system of this type, which can be utilized to enrich for the modified DNA by the methods of the disclosure. A comprehensive database of restriction enzymes, modifying enzymes, e.g. methylases, and sensitivity to modifications may be found at the New England Biolabs rebase site. Any of the Type I, II, III, or IV restriction modification systems provides for cleavage of DNA populations that can then be depleted by exonuclease treatment subsequent analysis.

Many restriction enzymes have corresponding methyltransferases that modify one or more of the bases in the recognition sequence, thereby protecting the host DNA from the action of the restriction enzyme. Many restriction enzymes are sensitive to methylation at bases other than those recognized by the cognate methylases. Sometimes, cleavage is blocked completely, but more often the rate of cleavage is affected and so depending upon the length of time of the digestion, or the amount of enzyme that is used, partial cleavage is often observed.

Known DNA modifications include, for example, glucosylated-hydroxymethylcytosine, N4-methylcytosine, 5-methylcytosine, 6-methyladenosine, 5-hydroxymethylcytosine, uracil, hydroxymethyluracil, 5-formylcytosine, 5-carboxylcytosine, queuosine, deoxyarchaeosine, and 7-deazaguanine.

DNA methylation. Certain bacterial strains methylate genomic DNA at specific sites. The differential cleavage of methylated vs. non-methylated DNA allows selective enrichment of the methylated DNA. Methylases of interest include, without limitation, Dam, Dcm, EcoBI, EcoKI and CpG methylases.

Dam methylase is encoded by the dam gene (Dam methylase), which transfers a methyl group from S-adenosylmethionine (SAM) to the N6 position of the adenine residues in the sequence GATC.

The Dcm methylase methylates the internal (second) cytosine residues in the sequences CCAGG and CCTGG at the C5 position.

The EcoKI methylase, M. EcoKI, modifies adenine residues in the sequences AAC (N6) GTGC and GCAC (N6) GTT.

The EcoBI methylase modifies adenine residues in the sequence TGA (N) & TGCT.

Two methylated motifs that are broadly prevalent in clinically relevant bacteria are 5′-RAATTY-3′ and 5′-GANTC-3′ (R=A or G; Y=C or T; N=any nucleotide). Unmethylated 5′-RAATTY-3′ is endonuclease-targeted by Apol and, in subset, by EcoRI (5′-GAATTC-3′). Unmethylated 5′-GANTC-3′ is endonuclease-targeted by Hinfl and, in subset, by Tfil. DNA from organisms that methylate these motifs resist the action of the listed endonucleases.

Restriction endonucleases. As discussed above, many restriction endonucleases are known and used in the art, and are readily available to one of skill in the art. In some embodiments, a endonclease of interest for use in the methods of the disclosure is PspBI (see Morgan et al. Appl Environ Microbiol. 1998 October; 64 (10): 3669-3673). PspGI is an isoschizomer of EcoRII and cleaves DNA before the first C in the sequence 5′ \hat{ }CCWGG 3′ (W is A or T). PspGI digestion can be carried out at different temperatures. The recognition sequence of PspGI is the same as that of the Dom methylase, which modifies the internal C at the cytosine-5 position in 5′ CCWGG 3′ sites.

EcoRII is a homodimeric type IIE restriction endonuclease. It recognizes the DNA sequence 5′CCWGG-(N)ₓ-CCWGG. The unspecific spacer (N)ₓ should not exceed 1000 bp. EcoRII is blocked by overlapping dcm methylation.

Mbol restriction enzyme recognizes \hat{ }GATC sites. Mbol is blocked by dam methylation. Isoschizomers include BfuCI, BssMI, BstKTI, BstMBI, DpnII, Kzo9I, NdeII, Sau3A.

Dpnl restriction enzyme recognizes and cleaves 5′-GATC-3′ sites that are dam methylated.

Distributive exonucleases. Exonucleases can be classified by the products of the reaction (mononucleotides vs. oligonucleotides) and whether released products contain 5′ or 3′ phosphate residues. Processive exonucleases will bind to the substrate and execute a series of hydrolysis events before dissociation. On the other hand, other exonucleases are “distributive”, with exonuclease molecules releasing only to be rebound or replaced by another exonuclease molecule a few or many times in the course of degrading a single target.

Distributive exonucleases include, for example, EcoX, ExoIII, T5 exonuclease, etc. T5 exonuclease catalyzes the degradation of nucleotides either from the 5′ termini or at nicks of linear or circular dsDNA in a 5′ to 3′ direction. This exonuclease also exhibits ssDNA endonuclease activity in the presence of magnesium ions, but will not degrade supercoiled dsDNA.

Digestion with the distributive exonuclease is performed for a period of time sufficient to distinguish between cleaved and uncleaved DNA, for example for at least about 10 minutes, at least about 15 minutes, at least about 20 minutes, and may be not more than about 1 hour.

The methods of the present disclosure may include sequencing enriched DNA, e.g. to identify the presence of a pathogen genome in a sample, or to obtain higher read coverage of potential pathogen genome of interest. Various methods and protocols for DNA sequencing and analysis are well-known in the art and are described herein. For example, DNA sequencing may be accomplished using high-throughput DNA sequencing techniques. Examples of next generation and high-throughput sequencing include, for example, massively parallel signature sequencing, polony sequencing, 454 pyrosequencing, Illumina (Solexa) sequencing with HiSeq, MiSeq, and other platforms, SOLID sequencing, ion semiconductor sequencing (Ion Torrent), DNA nanoball sequencing, heliscope single molecule sequencing, single molecule real time (SMRT) sequencing, MassARRAY®, and Digital Analysis of Selected Regions (DANSR™). See, e.g., Stein RA (1 Sep. 2008). “Next-Generation Sequencing Update”. Genetic Engineering & Biotechnology News 28 (15); Quail, Michael; Smith, Miriam E; Coupland, Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of lon torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012:1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge W A, Peters D G. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; international journal of clinical chemistry 2008; 387:55-8). Similarly, software programs for primary and secondary analysis of sequence data are well-known in the art.

Third generation sequencing is also of interest, which includes, for example, single molecule real time sequencing (SMRT), based on the properties of zero-mode waveguides (PacBio), Oxford Nanopore sequencing; Stratos Genomics; and the like. In some embodiments, high-throughput sequencing involves the use of technology available by Helicos BioSciences Corporation (Cambridge, Massachusetts) such as the Single Molecule Sequencing by Synthesis (SMSS) method. In some embodiments, high-throughput sequencing involves the use of technology available by 454 Lifesciences, Inc. (Branford, Connecticut) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours. In some embodiments, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication application Nos. 200401061 30; 20030064398; 20030022207; and Constans, A, The Scientist 2003, 17 (13): 36.

Library preparation, in the absence or presence of amplification, may be used to generate libraries for sequencing. The library preparation may include tagging with sites for sequencing primers.

In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read. Sequencing can be performed using nucleic acids described herein. Sequencing may comprise massively parallel sequencing.

In some embodiments, high-throughput sequencing of RNA or DNA can take place using AnyDot.chips (Genovoxx, Germany), which allows for the monitoring of biological processes. In particular, the AnyDot-chips allow for 10×-50× enhancement of nucleotide fluorescence signal detection. Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication application No. 20030044781 and 2006/0078937. The growing of the nucleic acid strand and identifying the added nucleotide analog may be repeated so that the nucleic acid strand is further extended and the sequence of the target nucleic acid is determined.

The methods disclosed herein may comprise amplification of DNA. Amplification may comprise PCR-based amplification. Alternatively, amplification may comprise nonPCR-based amplification. Amplification of the nucleic acid may comprise use of one or more polymerases. The polymerase may be a DNA polymerase. The polymerase may be a RNA polymerase. The polymerase may be a high fidelity polymerase. The polymerase may be KAPA HiFi DNA polymerase. The polymerase may be Phusion DNA polymerase. Amplification may comprise 20 or fewer amplification cycles. Amplification may comprise 20, 19, 18, 17, 16, 15, 14, 13, 12, 11, 10, or 9 or fewer amplification cycles. Amplification may comprise 18 or fewer amplification cycles. Amplification may comprise 16 or fewer amplification cycles. Amplification may comprise 15 or fewer amplification cycles.

Sequencing reads may be demultiplexed, and mapped to their corresponding genomes using steps of data analysis, which may be provided as a program of instructions executable by computer and performed by means of software components loaded into the computer. Such methods include aligning and mapping sequences to known genomes. The method may further comprise providing a computer-generated report comprising the characterization of genomes present in a sample.

Disclosed herein are systems for implementing one or more of the methods or steps of the methods disclosed herein. A computer system includes a central processing unit (CPU, also “processor” and “computer processor” herein), which can be a single core or multi core processor, or a plurality of processors for parallel processing. The system also includes memory (e.g., random-access memory, read-only memory, flash memory), electronic storage unit (e.g., hard disk), communications interface (e.g., network adapter) for communicating with one or more other systems, and peripheral devices, such as cache, other memory, data storage and/or electronic display adapters. The memory, storage unit, interface and peripheral devices are in communication with the CPU through a communications bus, such as a motherboard. The storage unit can be a data storage unit (or data repository) for storing data. The system is operatively coupled to a computer network with the aid of the communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network in some cases, with the aid of the system, can implement a peer-to-peer network, which may enable devices coupled to the system to behave as a client or a server.

The system is in communication with a processing system. The processing system can be configured to implement the methods disclosed herein. In some examples, the processing system is a nucleic acid sequencing system, such as, for example, a next generation sequencing system (e.g., Illumina sequencer, lon Torrent sequencer, Pacific Biosciences sequencer). The processing system can be in communication with the system through the network, or by direct (e.g., wired, wireless) connection. The processing system can be configured for analysis, such as nucleic acid sequence analysis.

Methods as described herein can be implemented by way of machine (or computer processor) executable code (or software) stored on an electronic storage location of the system, such as, for example, on the memory or electronic storage unit. During use, the code can be executed by the processor. In some examples, the code can be retrieved from the storage unit and stored on the memory for ready access by the processor. In some situations, the electronic storage unit can be precluded, and machine-executable instructions are stored on memory.

Read mapping is the process to align the reads on reference genomes, taking as input a reference genome and a set of reads, and aligning reads on the reference genome. Many programs for mapping are available in the art, including, for example, Bowtie2. Public domain databases, such as NCBI GenBank and EMBL, contain sequences, including complete genomes, of multiple species.

In one embodiments, disclosed herein is a computer-implemented system for characterizing a sample with respect to the presence of a genome of interest, where the samples are prepared by the methods disclosed herein and sequenced. The computer-implemented system may comprise (a) a digital processing device comprising an operating system configured to perform executable instructions and a memory device; and (b) a computer program including instructions executable by the digital processing device, the computer program comprising (i) a first software module configured to receive data pertaining to DNA sequencing; (ii) a second software module configured to map the DNA to known reference genomes.

The methods disclosed herein may comprise generating libraries from the enriched DNA, by using recombinant methods known in the art.

The term “diagnosis” is used herein to refer to the identification of a molecular entity in a sample.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

An “effective amount” means the amount of a compound or enzyme that, when contacted with a substrate, is sufficient to effect a desired treatment.

The term “unit dosage form,” as used herein, refers to physically discrete units suitable as unitary dosages for achieving a desired effect, each unit containing a predetermined quantity of a compound or enzyme calculated in an amount sufficient to produce the desired effect. The specifications for unit dosage forms depend on the particular compound or enzyme employed and the effect to be achieved, and the pharmacodynamics associated with each compound in the host.

A “physiologically acceptable excipient,” means an excipient, diluent, carrier, and adjuvant that are useful in preparing a composition that are generally safe, non-toxic and neither biologically nor otherwise undesirable.

Kits may be provided. Kits may comprise, for example, one or a cocktail of endonucleases, for example a cocktail of enzymes specific for one or more different recognition sites, including without limitation where at least one enzyme is blocked by Dcm modification and at least one enzyme is blocked by Dam methylation; and a distributive enonuclease. Kits may further comprise buffers and reagents suitable for carrying out digestions; reagents for sequencing, instructions for use; and the like. Kits may also include tubes, buffers, etc., and instructions for use.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Restriction Endonuclease-Based Modification-Dependent Enrichment (REMODE) of DNA for Metagenomic Sequencing

Metagenomic sequencing is a swift and powerful tool to ascertain the presence of an organism of interest in a sample. However, sequencing coverage of the organism of interest can be insufficient due to an inundation of reads from irrelevant organisms in the sample. Here, we report a nuclease-based approach to rapidly enrich for DNA from certain organisms, including enterobacteria, based on their differential endogenous modification patterns. We exploit the ability of taxon-specific methylated motifs to resist the action of cognate methylation-sensitive restriction endonucleases that thereby digest unwanted, unmethylated DNA. Subsequently, we use a distributive exonuclease or electrophoretic separation to deplete or exclude the digested fragments, thus, enriching for undigested DNA from the organism of interest. As a proof-of-concept, we apply this method to enrich for the enterobacteria Escherichia coli and Salmonella enterica by 11- to 142-fold from mock metagenomic samples and validate this approach as a versatile means to enrich for genomes of interest in metagenomic samples.

Pathogens that contaminate the food supply or spread through other means can cause outbreaks that bring devastating repercussions to the health of a populace. Investigations to trace the source of these outbreaks are initiated rapidly but can be drawn out due to the labored methods of pathogen isolation. Metagenomic sequencing can alleviate this hurdle but is often insufficiently sensitive. The approach and implementations detailed here provide a rapid means to enrich for many pathogens involved in foodborne outbreaks, thereby improving the utility of metagenomic sequencing as a tool in outbreak investigations. Additionally, this approach provides a means to broadly enrich for otherwise minute levels of modified DNA which may escape unnoticed in metagenomic samples.

Here we describe and implement REMODE: Restriction Endonuclease-based Modification-Dependent Enrichment of DNA, an approach that rapidly and cost-effectively enriches for DNA from E. coli and S. enterica in metagenomic samples. We rely on the presence of Dam and Dcm methyltransferases in E. coli and S. enterica and the near complete methylation of all instances of their target motifs in E. coli and S. enterica. These methyltransferases methylate 5′-GATC-3′ and 5′-CCWGG-3′ respectively (methylated base underlined; W=A or T. We employ the highly specific action of methylation-sensitive restriction enzymes Mbol (5′-GATC-3′), PspGI and EcoRII (both 5′-CCWGG-3′) that cleave only unmethylated instances of the motif. When applied to a mixed population of DNA that is unmethylated or methylated at these motifs, we observe a segregation of DNA into either short or long, genomic-length fragments respectively. Finally, we select for the longer fragments of DNA either using electrophoretic separation or by taking advantage of the highly distributive nature of the T5 exonuclease. When applied to a reaction with different distributions of long and short DNA, electrophoretic separation provides a clean size separation, albeit requiring an additional gel isolation step, while the T5 exonuclease reaction is a cost-effective approach that can be adjusted to rapidly deplete short DNA in a same tube reaction. We observe a 11- to 142-fold enrichment of E. coli and S. enterica DNA in a metagenomic sample relative to an untreated version of the same sample. This method can be extended to other Dam and Dcm methylated organisms and can be extrapolated to organisms with other methylation patterns.

### Results

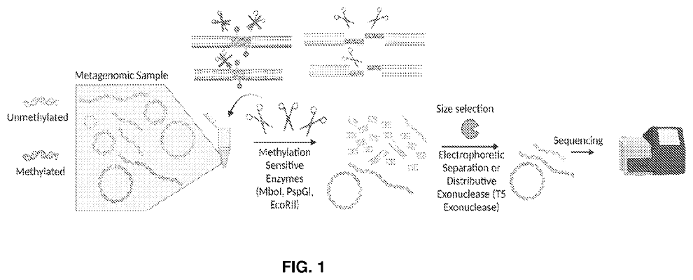

FIG. 1 provides an overview of the restriction-enzyme-based scheme that we have used to enrich for DNAs methylated at defined sites. As a proof of principle, we elected to test this approach with DNA from organisms readily available in the laboratory and that we knew were Dam and Dcm methylated (E. coli) or unmethylated (C. elegans).

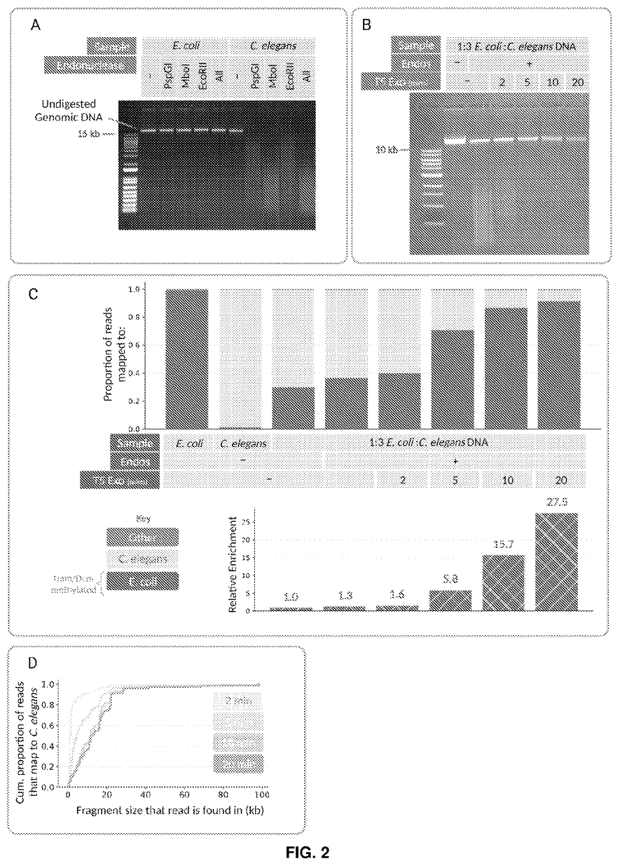

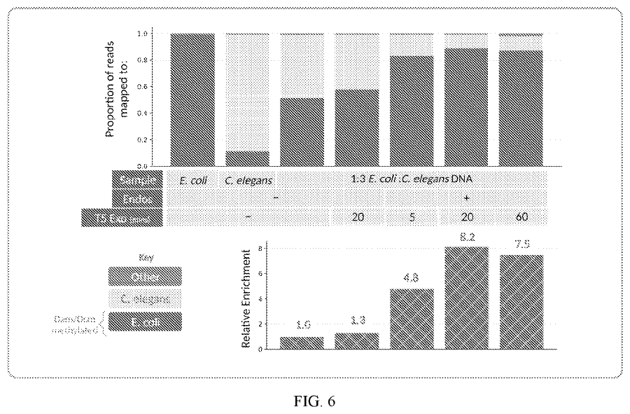

Genomic DNA from TOP10 E. coli and N2 C. elegans was prepared and treated with restriction endonucleases Mbol, PspGI and EcoRII. The DNA was found to be either resistant or susceptible to the action of these endonucleases, respectively (FIG. 2A). A 1:3 mixture (by mass) of genomic DNA from E. coli and C. elegans was prepared as a stand-in for a metagenomic sample. After treatment with the endonucleases, the sample was treated with the distributive T5 exonuclease for 2, 5, 10 or 20 minutes. When treated for five minutes, or beyond, shorter fragments were substantially depleted from the sample, while longer fragments were retained (FIG. 2B). The preferential retention of longer fragments was as expected given the timing of the reaction and rates of terminus-degrading exonuclease activity. When sequenced on an Illumina MiSeq, a progressive enrichment of the proportion of reads that mapped to E. coli was observed (FIG. 2C). Relative enrichment values were calculated as the ratio of the number of E. coli reads to C. elegans reads in a treated sample divided by the ratio in an untreated sample. With 20 minutes of exonuclease treatment, a 27.5 fold enrichment was observed (FIG. 2C). Longer T5 treatment did not result in greater enrichment of E. coli reads in this mixture (FIG. 6). To determine where the remaining proportion of C. elegans reads were originating from, each C. elegans read was assigned the theoretical length of the restriction fragment it came from in an in-silico digestion of the C. elegans genome. The cumulative distribution of these lengths was plotted for each T5 exonuclease time point (FIG. 2D) and many C. elegans reads, as expected, originated from regions greater than 10 kb.

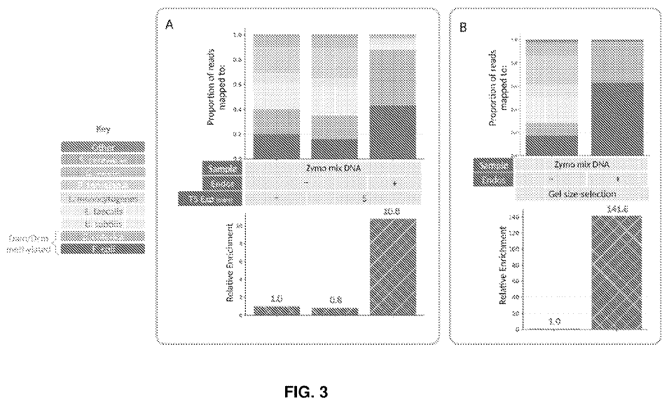

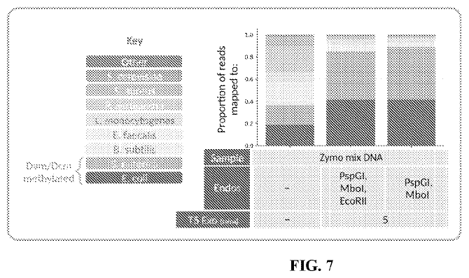

To assay a more complex but standardized sample, we performed this treatment on ZymoBIOMICS microbial community standard high molecular weight DNA (hereafter referred to as Zymo mix). This is a mixture of genomic DNA from one yeast and seven bacteria—of which two (E. coli and S. enterica) are Dam and Dom methylated. Upon PspGI, Mbol and EcoRII endonuclease and T5 exonuclease treatment, an average of a 10.8-fold relative enrichment of E. coli and S. enterica DNA was observed (FIG. 3A). DNA from these two species composes 28% of the untreated Zymo mix according to the manufacturer (Zymo Research). However, we found that roughly 35 to 40% of reads from an untreated sample map to the E. coli and S. enterica genomes. This is likely due to our transposition-based sequencing library construction methods which have a bias against GC-rich genomes. Additionally, it was observed that the enrichment worked just as well without EcoRII, which thus may be omitted (FIG. 7). However, to maintain experimental consistency, EcoRII was used for all following experiments.

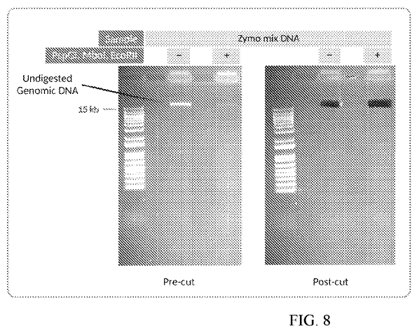

The addition of the T5 exonuclease acts to select for long fragments of DNA rapidly (5 to 20 mins). This approach has the advantage of a low cost and can be performed in the same tube as the endonuclease treatment. We were curious how this might compare to the gold standard of electrophoretic size selection (agarose gel electrophoresis). Endonuclease untreated and treated Zymo mix DNA samples were resolved on a gel alongside each other (FIG. 8). Due to the size exclusion limit of a 1% gel, all fragments greater than ˜15 kb (highest band of the ladder) comigrate as a single band. In the treated sample, a smear throughout the lane is observed, however high molecular weight DNA originating from E. coli and S. enterica is found well above the 15 kb marker. Both the untreated and the treated high molecular weight bands were extracted from the gel and sequenced. Virtually all of the reads from the treated sample mapped to E. coli and S. enterica, providing a substantial relative enrichment of 141.8-fold (FIG. 3B). Electrophoretic size selection therefore proves to be an effective way of separating digested fragments from undigested fragments. However, it comes at the cost of time and money over a T5 exonuclease size selection.

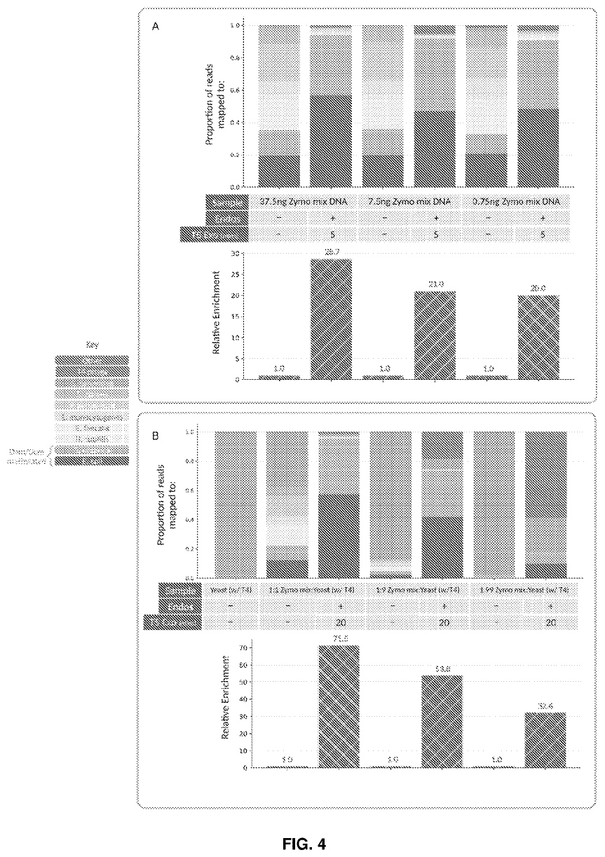

Next, to test the dynamic range of exonuclease-based enrichment, we titrated down the input Zymo mix DNA amount from the initial value of 75 ng to 37.5 ng (1/2), 7.5 ng (1/10) and 0.75 ng (1/100). In all cases, we observed on average a greater than 20-fold relative enrichment of E. coli and S. enterica DNA (FIG. 4A). While the protocol continued to be effective for vanishingly small amounts of input DNA, we questioned how the enrichment varied when the ratio of Dam/Dcm methylated DNA to unmethylated DNA was altered. To test this, the Zymo mix DNA was mixed with Saccharomyces cerevisiae genomic DNA in a 1:1, 1:9 and 1:99 ratio. We observed a robust enrichment of E. coli and S. enterica DNA at all ratios, with average enrichment ranging from 32.4- to 71.5-fold (FIG. 4B).

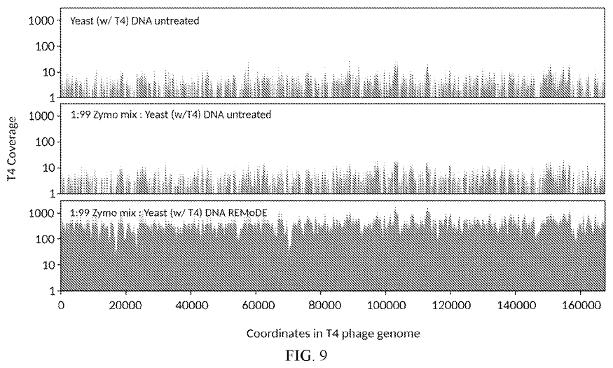

There was a surprising enrichment of a class of reads that did not seem to map to any of the genomes present in the Zymo mix or S. cerevisiae. This class of reads was reproduced upon replicate experiments. These reads were assembled into contigs using SPADES. The largest, most prevalent contig from these unmapped reads mapped to E. coli bacteriophage T4 using BLAST (shown in FIG. 4B). Upon closer inspection, we realized some phage T4 DNA was unintentionally included with the S. cerevisiae DNA (FIG. 9). Enrichment of T4 DNA is expected because it contains hydroxymethyl cytosine, usually glucosylated, in place of cytosine. This allowed the T4 DNA to resist cleavage by PspGI, Mbol and EcoRII and was therefore selected for during T5 exonuclease treatment, serving as a fortuitous positive control (FIG. 9). This points towards the ability to use this tool as way to discover genomes in metagenomic samples that are substantially modified or contain non-canonical bases (see discussion).

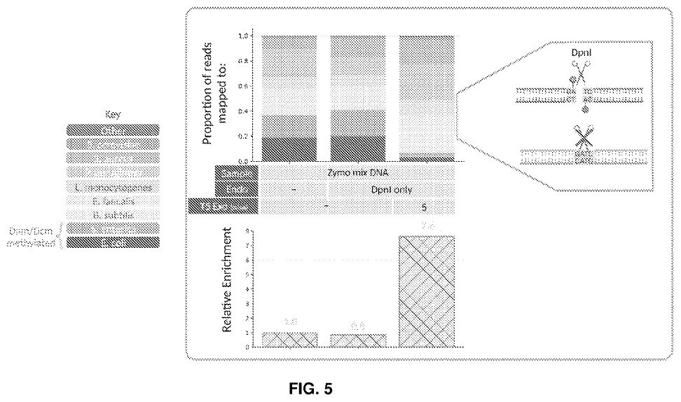

Finally, we asked whether a parallel protocol could be used for selective enrichment of unmodified DNA. Dpnl is a restriction endonuclease that selectively cleaves at Dam sites that are methylated (unlike Mbol which cleaves at Dam sites that are unmethylated). Accordingly, Dpnl can be used to deplete E. coli and S. enterica DNA in a metagenomic sample. When Dpnl was applied to the Zymo mix, a 7.6-fold relative enrichment of non-Dam methylated DNA was observed as compared to the untreated control (FIG. 5).

### DISCUSSION

In this work we have described and implemented a novel approach, REMODE, to enrich metagenomic samples for DNA from organisms of interest based on their specific patterns of DNA modification. While differential methylation has been used to obtain enriched sequence datasets in the past, the technical approaches have involved binding and release steps with high complexity in terms of reagents and protocols. Applying restriction enzyme cleavage followed by exonuclease- or gel-based size selection, we obtained remarkable enrichments with only limited manipulation.

The approach provided herein specifically includes methods to selectively enrich DNA of organisms that contain Dam and Dom systems. These methyltransferases are found in many members of the Gammaproteobacteria phyla including E. coli and S. enterica. Many pathogenic food outbreaks have been caused by species from the Gammaproteobacteria phyla. Various food and agricultural safety applications require high sequencing coverage of the outbreak strain to confidently obtain identifying SNPs for an outbreak source (optimal coverages may be as high as 50×). Such coverage allows potential matching of the agricultural source with contaminated foods, providing an opportunity to accurately restrict further outbreak from the source, while avoiding interference with supply chains uninvolved in an outbreak.

Sequencing approaches lend tremendous specificity and sensitivity to detection and characterization of potential pathogens. However, challenges in utilization of a sequencing approach can arise in that metagenomic samples from both environmental and clinical sources generally contain irrelevant prokaryotic and eukaryotic DNA that far exceeds that of the pertinent strain and therefore obtaining high coverage WGS can prove difficult. This approach proves especially useful in metagenomic analyses such as these. In our experiments, we observe enrichment of E. coli and S. enterica DNA ranging from 11-fold to 142-fold with a broad dynamic range for input DNA amount. Additionally, the method has been developed such that the enrichment can be performed in a single tube and completed within one-and-a-half hours.

The presence of high molecular weight input DNA is necessary for effective segregation of protected and unprotected fragments. The concentration of input DNA when using T5 exonuclease as a size-selection technique as optimal exonuclease activity is dependent on both concentration of DNA and reaction time. When time, cost and highly parallel processing is not of concern and substantial enrichment is, gel electrophoresis may be as the technique of choice for size selection.

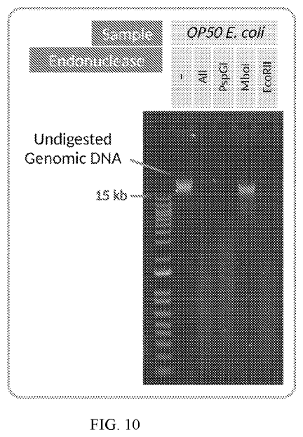

Strains with different patterns of modification exist for some species. For example, B strain E. coli have lost their ability for Dom methylation, likely in the laboratory. We encountered this as we tried to enrich for OP50 E. coli DNA and found, instead, that it was digested by PspGI and EcoRII indicating that it was not Dcm methylated (FIG. 10). OP50 is derived from B strain E. coli. Also, to be noted is that while Dam methyltransferases are indeed widespread (though not ubiquitous) within Gammaproteobacteria, Dcm methyltransferases may be confined to genera closely related to Escherichia. The piecemeal distribution of Dcm methylation may serve as an advantage in REMODE applications depending on the organismal DNA to be enriched for. Since the Dam motif is shorter than the Dom motif, it is found more frequently in any given genome. Hence, Mbol contributes most to the segregation of methylated and unmethylated DNA at these sites as compared to PspGI and EcoRII (FIG. 2B) suggesting that an Mbol only digestion would be sufficient to achieve strong enrichment. Indeed, it has also been found that Dam serves a core function for gene expression of virulence factors and that Dam inhibition attenuates virulence and pathogenicity in Dam bacteria in vivo. Pathogenic strains leading to outbreak such as O157: H7 have been found to contain the genes for both Dam and Dcm.

Extension of REMODE for other applications. Dam and Dcm systems extend to clinically relevant organisms beyond E. coli and S. enterica that benefit from whole genome sequencing for tracing purposes. For example, Vibrio cholerae (causes cholera), Yersinia pestis (causes plague), Legionella pneumophila (causes Legionnaire's disease), and Klebsiella pneumoniae (causes pneumonia) are either known or predicted to methylate their Dam sites. Some eukaryotic viruses are also known to harbor methyltransferases. For example, the Melbournevirus of the giant virus family Marseilleviridae is also Dam methylated.

The principle of enrichment using restriction enzymes and an exonuclease need not only extend to Dam and Dcm methylated DNA. As shown, unmodified DNA can be enriched using Dpnl and this paradigm can be applied more broadly by taking advantage of the vast catalogue of restriction enzymes. Among others, there are two methylated motifs that are broadly prevalent in clinically relevant bacteria: 5′-RAATTY-3′ and 5′-GANTC-3′ (R=A or G; Y=C or T; N=any nucleotide). Unmethylated 5′-RAATTY-3′ is endonuclease-targeted by Apol and, in subset, by EcoRI (5′-GAATTC-3′). Unmethylated 5′-GANTC-3′ is endonuclease-targeted by Hinfl and, in subset, by Tfil (5′-GAWTC-3′; W=A or T). DNA from organisms that methylate these motifs resist the action of the listed endonucleases.

One such clinically relevant genus is Campylobacter (5′-RAATTY-3′) which is known to cause widespread foodborne illness across the globe, and is estimated to cause more than 1.5 million infections per year in the US, and close to nine million in the European Union. Campylobacter is often associated with the contamination and microbiome of poultry and wild birds. Apol and EcoRI can enrich for these bacteria in metagenomic samples.

Another scenario where enrichment of pathogenic DNA for whole genome sequencing purposes is particularly useful is in the case of nosocomial infections (infections that originate in the hospital). These are often spread through patient-to-patient contact or patient-to-surface-to-patient contact and need to be traced to origin. Such is the case, for example, for the opportunistic pathogen Acinetobacter baumanii (5′-RAATTY-3′) which initially cropped up in medical military facilities and quickly spread to civilian medical facilities by way of infected soldiers being transported through them. This is in addition to bacteria such as Klebsiella pneumoniae that may use the Dam/Dcm systems described above.

Mycoplasma bovis (5′-GANTC-3′) is known to infect cattle and has resulted in an estimated loss of $108 million in the US annually. Similarly, species abortus, melitensis and suis of the genus Brucella (5′-GANTC-3′) are known to cause Brucellosis in livestock. This method may accordingly prove useful in disease tracking within livestock settings.

Oliveira and Fang (2021) Trends Microbiol 29:28-40 detail the presence and distribution of different methylated motifs across clades of bacteria which can be used to select appropriate restriction enzymes for an organism of interest.

REMODE as a discovery tool. Of interest in understanding the results of REMODE assays are the characteristics of DNA fragments from non-methylated organisms that remain after digestion and are represented in the sequencing data. Several features could result in the survival of these fragments including a lack of restriction sites in long stretches of a genome, circular DNA (that does not contain the corresponding restriction sites and is insusceptible to exonuclease degradation), or protection of DNA ends on linear fragments due to specific chemical structures or linkage to a terminal protein. Likewise novel DNA modifications (or damaged bases) could render some or all fragments from a given experimental source resistant to the initial endonuclease digestions.

Restriction-modification systems evolved such that a host cell's restriction enzymes would be unable to digest host DNA due to the presence of protective modifications which infecting phage DNA would not have. As such, Type II restriction enzymes are very specific to their cognate restriction sites but are blocked by these modifications. This proves a useful method to distinguish modified DNA from unmodified DNA. In some cases, these enzymes are unable to cleave DNA with other modifications within the restriction site and not just with the modification associated with the corresponding restriction-modification system. Indeed, certain phage (T4, S2-L etc.) modify all instances of a base (C in T4, A in S2-L) in their genome and when purified DNA from these phages is treated with restriction enzymes, the DNA withstands the action of these enzymes. This is why a substantial enrichment of T4 DNA was observed in our experiments when there was an inadvertent inclusion of T4 DNA in our yeast DNA sample. REMODE can be used to screen environmental samples for DNAs resistant to the action of a selection of endonucleases. Such sequences may comprise non-canonical bases or modifications. This DNA can then be sequenced either by standard short read sequencing (e.g. Illumina) or by methods conducive to distinguishing modified residues such as Oxford Nanopore or PacBio Single Molecule Real Time (SMRT) sequencing.

### Methods

Genomic DNA preparation. Typical methods for genomic DNA preparation should function well for REMODE as long as caution is taken to limit extensive shearing of purified DNA. The methods of DNA purification employed in this study were relatively standard and we have extensively detailed these below for reproducibility.)

E. coli. Protocol adapted from Green and Sambrook et al. 1.5 mL of an overnight culture (2× TY media) of Top10 E. coli was centrifuged at 5,000 RCF at room temperature for 30 seconds and the supernatant removed by aspiration. 400 μL of 10 mM Tris 1 mM EDTA (TE) buffer at pH 8.0 was added to the tube and the bacterial pellet was resuspended via gentle vortexing. 50 μL of 10% SDS and 50 μL of Proteinase K (20 mg/mL in TE, pH 7.5) was added to the tube and left to incubate at 37° C. for 1 hour. The digested lysate was pipetted up and down three times with a p1000 pipette to reduce viscosity. 500 μL of a 1:1 mixture of phenol:chloroform (phenol equilibrated with 10 mM Tris-HCl, pH 8.0) was added to the tube and pipetted up and down multiple times to mix. The mixture was then transferred to a 2 mL phase lock light tube (5 PRIME 2302800) and centrifuged at 16,000 RCF at room temperature for 5 minutes. The aqueous phase was transferred to a new phase lock tube and the 1:1 phenol: chloroform extraction was repeated. The aqueous phase was then extracted twice with 500 μL chloroform. The suspension was transferred to a fresh microcentrifuge tube and 25 μL of 5M NaCl followed by 1 mL of ice-cold 95% ethanol was added. The mixture was pipetted up and down multiple times and then centrifuged at 21,000 RCF at 4° C. for 10 minutes. The supernatant was carefully removed with a pipette and left to dry for 10 minutes. The damp-dry pellet was dissolved in 100 μL of TE. 2.5 μL of RNaseA (10 mg/mL; Thermo Scientific EN0531) was added to the solution, mixed, and left to incubate for 30 minutes at 37° C. 40 μL of 5M ammonium acetate and 250 μL of isopropanol were added to the mixture, mixed with a pipette and left to incubate at room temperature for 10 minutes with the cap closed. The tube was centrifuged at 21,000 RCF at room temperature for 10 minutes. The pellet was washed twice with 70% ethanol and then the ethanol was aspirated carefully with a pipette. The tube was left to dry for 10 minutes. The pellet was then dissolved in 100 μL TE (pH 8.0) and left to incubate overnight at 37° C. for complete dissolution. The concentration was determined using Qubit BR dsDNA reagents and a Qubit 2.0 fluorometer.

C. elegans. Worms from three 60 mm×15 mm starved plates of N2-strain (PD1074) C. elegans were collected by washing them off the plate with 1.5 mL of 50 mM NaCl and into a 1.5 mL tube. The tube was centrifuged for 40 seconds at 400 RCF at room temperature. Approximately 1200 μL of the supernatant was aspirated out, leaving roughly 300 μL of worms and solution. In a fresh 1.5 mL tube 1.2 mL of 50 mM NaCl containing 5% sucrose was added. The remaining 300 μL of the worms and solution was mixed and layered over the sucrose cushion. The tube was centrifuged for 40 seconds at 400 RCF. The supernatant was removed and the pellet was washed with 1.5 mL of 50 mM NaCl. The tube was centrifuged again for 40 seconds and the supernatant removed. 450 μL of Worm Lysis Buffer (0.1M Tris pH 8.5, 0.1M NaCl, 50 mM EDTA and 1% SDS) was added to the tube along with 20 μL of proteinase K (20 mg/mL). The tube was gently vortexed. The tube was left to incubate at 62° C. for 45 minutes and vortexed four to five times throughout the incubation. 500 μL of phenol was added to the tube, mixed thoroughly by pipetting up and down, and transferred to a phase lock light tube. The tube was centrifuged for 5 minutes at 16,000 RCF. The aqueous phase was transferred to a new phase lock tube and extracted with 500μLs of 1:1 phenol: chloroform. Finally, the aqueous phase, again, was extracted with 500μLs of chloroform and transferred to a fresh 1.5 ml tube. 80 μL of 5M ammonium acetate was added to the solution. 1 mL of ethanol was added to the tube and mixed thoroughly by pipetting. The tube was then centrifuged for 5 minutes at 21,000 RCF at room temperature and the pellet was washed once with 0.5 mL of ethanol and centrifuged again. The ethanol was aspirated out and the pellet was left to dry for 10 minutes at room temperature after which 25 μL of TE (pH 8.0) was used to resuspend it. The concentration was determined using Qubit BR dsDNA reagents and a Qubit 2.0 fluorometer. Note that RNase was not used in this preparation and thus downstream experiments with C. elegans contain C. elegans RNA, however DNA was RNaseA treated before loading onto gel in FIG. 2A.

S. cerevisiae. 4 mL of an overnight S288C yeast culture (YPD media) was pelleted at 16,000 RCF for 1 minute and resuspended in 250 μl of Breaking Buffer (2% (v/v) Triton X 100, 1% (w/v) SDS, 100 mM NaCl, 10 mM Tris base pH 8, 1 mM EDTA). Approximately to the volume of 200 μL of 0.5 mm glass beads was added to the mixture as well as 500 μL of 1:1 phenol: chloroform. The tube was vortexed, at max speed, at 4° C. for 10 minutes. It was then centrifuged at 16,000 RCF at 4° C. for 10 minutes. 400 μL of the aqueous phase was transferred to a fresh 1.5 ml tube. 1 μL of RNase A (10 mg/mL) was added to the mixture and it was left to incubate at 37° C. for 10 mins. 750 μL of 1:1 phenol: chloroform was added to the tube and mixed well with a pipette. The solution was transferred to a 2 mL phase lock light tube and centrifuged for 5 minutes at 16,000 RCF at room temperature. The aqueous phase was then transferred to a fresh 1.5 mL tube and 65 μL of 3M sodium acetate was added to the tube. 1 mL of ice-cold ethanol was added to the tube, mixed, and left to incubate for 10 minutes at −20° C. The tube was centrifuged for 10 minutes at 21,000 RCF. The supernatant was carefully aspirated out and the pellet was washed with 1 mL of ice-cold 70% ethanol. The tube was spun again for 10 minutes at 21,000 RCF at 4° C. The supernatant was carefully aspirated out and the pellet was left to dry for 10 minutes. It was then resuspended in 20 μL of TE. The concentration was determined using Qubit BR dsDNA reagents and a Qubit 2.0 fluorometer.

ZymoBIOMICS MCS-HMW DNA. ZymoBIOMICS MCS-HMW DNA (Zymo D6322; “Zymo mix”) was obtained from Zymo Research. The concentration was determined using Qubit BR dsDNA reagents and a Qubit 2.0 fluorometer and found to be slightly lower than the manufacturer specifications (78 ng/μL as opposed to 100 ng/μL). For all following experiments, the Qubit-measured concentration was used instead of the manufacturer provided one. Zymo Research reports that the standard contains DNA >50 kb in size.

Endonuclease and exonuclease treatment. For initial experiments, a substantial amount of DNA was used (750 ng) as input and it was later found that the input could be decreased manifold. In the Zymo mix experiments, 75 ng of input DNA was used.

Both PspGI and EcoRII target Dcm sites and were used in these experiments. The redundancy is due to both enzymes requiring conditions that were inconvenient: PspGI has a high optimal temperature which is 75° C. that could be detrimental to the nucleic acids in the sample and EcoRII requires the presence of two Dcm sites in close proximity for cleavage. Hence, both enzymes were used at more convenient but suboptimal conditions: a 50° C. incubation for PspGI and an additional 30 min incubation (1 hr total) of EcoRII. However, as shown in FIG. 7, EcoRII may be omitted for seemingly no loss in fold enrichment with the Zymo mix.

The enzyme concentrations, conditions and incubation times described here may be modified to user specifications. Optimal T5 exonuclease concentration and incubation time may rely, among other factors, on the amount of DNA, the number of available DNA fragment termini, and the median length of DNA fragments in any given reaction. Ideally, time points for T5 exonuclease incubation should be taken for every uncharacterized sample as an extended incubation may result in overdigestion of DNA and limited enrichment of the genome of interest.

E. coli and C. elegans mixture. To set up 37.5 μL reactions, 1:3 mixtures of E. coli and C. elegans DNA were made by mixing 187.5 ng of E. coli genomic DNA with 562.5 ng of C. elegans genomic DNA in 8-strip PCR tubes. A volume of ultrapure water needed to make the reaction up to 37.5 μL after the addition of rCutsmart (NEB B6004S) and PspGI (NEB R0611) was added to each reaction followed by 3.75μL of 10× rCutsmart buffer and 0.6 μL (6U) of PspGI. The tubes were mixed via gentle vortexing after every step. Each mixture was incubated at 50° C. for 30 minutes after which 0.6 μL (3U) of Mbol (NEB R0147) was added to each reaction. Each mixture was incubated at 37° C. for 30 minutes after which 0.94 μL of 2M NaCl was added to each reaction (to bring the NaCl concentration to roughly 50 mM which is optimal for EcoRII (Thermo Scientific ER1921)). Then, 0.6 μL (6U) of EcoRII was added to each reaction. The mixture was incubated at 37° C. for 1 hour. The tubes were put on ice and 0.4 μL (4U) of T5 exonuclease (NEB M0663) was added to each reaction and incubated for 2, 5, 10 or 20 minutes at 37° C. and immediately quenched with 8 μL 6× NEB purple loading dye (NEB B7024S) supplemented with 6 mM EDTA (to make the total EDTA concentration in the stock tube 66 mM). 12 μL of the mixture was resolved on a 1% agarose gel run at 140V for 40 minutes. 74 μL of ultrapure water was added to the remaining sample (to make up to 100 μL total volume) and each reaction was then purified using the Zymo Genomic Clean and Concentrate kit (Zymo D4011). The DNA was eluted with 10 mM Tris buffer heated to 63° C. and incubated for between two to five minutes. The concentration was determined using Qubit HS dsDNA reagents and a Qubit 2.0 fluorometer. For control reactions, enzymes were replaced with an equal volume of ultrapure water at the appropriate point in the protocol.

ZymoBIOMICS MCS-HMW (Zymo mix). For the experiment in FIG. 3A, 75ng of Zymo mix DNA was used. A volume of ultrapure water needed to make the reaction up to 37.5 μL after the addition of rCutsmart and PspGI was added to each reaction followed by 3.75 μL of 10× rCutsmart buffer and 0.6 μL (6U) of PspGI. The tubes were mixed via gentle vortexing after every step. Each mixture was incubated at 50° C. for 30 minutes after which 0.6 μL (3U) of Mbol was added to each reaction. Each mixture was then incubated at 37° C. for 30 minutes after which 0.94 μL of 2M NaCl added to each reaction (to bring the NaCl concentration to roughly 50 mM which is optimal for EcoRII). Then, 0.6 μL (6U) of EcoRII was added to each reaction. The mixture was incubated at 37° C. for 1 hour. Note that as shown in FIG. 7, this step may be omitted and after incubation with Mbol, the reaction may proceed directly to T5 exonuclease digestion. The tubes were put on ice and 0.4 μL (0.4U) of T5 exonuclease diluted 1:10 in 1× NEBuffer 4 was added to each reaction and incubated for 5 minutes at 37° C. and immediately quenched with 8 μL 66 mM EDTA. The tubes were vortexed. 52 μL of TE was added to each reaction to make up to 100 μL total volume and purified using the Zymo Genomic Clean and Concentrate kit. The DNA was eluted with 15 μL of 10 mM Tris buffer heated to 50° C. and incubated for two to five minutes. This experiment was done in biological duplicate.

For the experiment in FIG. 3B, the same protocol was followed as in FIG. 3A except after the EcoRII incubation, 8 μL of NEB Purple Loading Dye was added and the samples were loaded into a 1% agarose gel and run for 45 minutes at 120V. The high molecular weight bands were excised with a razor and dissolved in 3 volumes of Zymo Agarose Dissolving Buffer (Zymo D4001) at 50° C. They were then processed through the Zymo Genomic Clean and Concentrate kit as in FIG. 3A excluding the step of the addition of ChIP DNA binding buffer. This experiment was done in biological duplicate and one of the duplicates was prepared from a previous experiment.

For the experiment in FIG. 4A, the same protocol was followed as in the experiment in FIG. 3A save for using either 37.5, 7.5 and 0.75 ng of input Zymo mix DNA.

For the experiment in FIG. 4B, different ratios of Zymo mix DNA and S. cerevisiae DNA were mixed together 1:1 (37.5 ng: 37.5 ng), 1:9 (7.5 ng: 67.5 ng) and 1:99 (0.75 ng: 74.25 ng) and the same protocol was followed as in FIG. 3A save for the T5 exonuclease incubation being 20 minutes instead of 5. This experiment was done in biological duplicate.

For the experiment in FIG. 5, the same protocol was followed as in FIG. 3A, save for a 1 hour incubation with 0.3 μL (3U) of Dpnl (NEB R0176) at 37° C. instead of using PspGI, Mbol or EcoRII.

FIGS. 3A (1 replicate), 5 and 7 plot the same untreated Zymo mix control data since these were performed in the same experiment. Additionally, FIG. 3A (1 replicate) and 7 plot the same PspGI, Mbol, EcoRII and T5 exonuclease treated data since these were performed in the same experiment.

Library Preparation and Miseq Sequencing. Nextera XT (Illumina FC-131-1024) library preparation was used to build sequencing libraries for all experiments. One-third of the recommended volumes of the manufacturer protocol were used i.e. 3.33 μL of Tagmentation buffer, 1.67 μL of 0.2 ng/μL DNA, 1.67 μL of Tn5 mix, 1.67 μL of the neutralizing buffer, 1.67 μL of each index followed by 5 μL of the polymerase mix. The transposition incubation was done at 37° C. for 5 minutes. Amplification was performed as in the Nextera XT protocol with 12 cycles of amplification. The amplified libraries were resolved on a gel and DNA of the range of 300 to 600 bp was excised for gel recovery using the Zymo gel extraction kit (Zymo D4007). Concentrations of DNA were determined with Qubit HS dsDNA reagents and a Qubit 2.0 fluorometer. The libraries were pooled and sequenced on an Illumina MiSeq sequencer using a MiSeq Reagent Kit v3 (MS-102-3001); 78 cycle, paired-end.

Data Analysis. Reads were demultiplexed on the Illumina MiSeq using the MiSeq Reporter. The resulting reads were mapped to their corresponding genomes via Bowtie2 version 2.4.5 using default settings. The reference file (FASTA format) for each experiment contained the genomes of each organism whose DNA was used in that experiment. The reference file was organized such that each chromosome or plasmid from every genome was given a header name unique to that species. Reads that mapped to each species were counted by parsing through the SAM file output by Bowtie2 and first binning each aligned read to its corresponding species in a Python3 list and then counting the elements of that list. The proportion of reads mapped to a particular species was obtained by dividing against the total number of aligned reads. The data were plotted using matplotlib in Python3 on Jupyter Notebook. Relative enrichment was calculated as follows:

$$\frac{\begin{matrix}\begin{matrix}( Proportion of reads from Dam and \\ Dcm methylated D N A in sample ) \div \end{matrix} \\ ( Proportion of reads from unmethylated D N A in sample )\end{matrix}}{\begin{matrix}\begin{matrix}( Proportion of reads from Dam and \\ Dcm methylated D N A in control ) \div \end{matrix} \\ ( Proportion of reads from unmethylated D N A in control )\end{matrix}}$$

Genome sequences. For E. coli and C. elegans experiments, genome assemblies GCA_000005845.2 (GenBank) and UNSB01000000 (European Nucleotide Archive) were used respectively. These genomes were combined into a single FASTA file used as a reference for Bowtie2 and the alignments were output as SAM files.

For Zymo mix experiments, genome assemblies were obtained from the protocol of this reagent. The genomes were combined into a single file. Since the assembly that was included for S. cerevisiae was heavily discontiguous and since the S288C strain of S. cerevisiae was used in the experiment for FIG. 4B, the provided S. cerevisiae was instead replaced with the latest assembly available on the Saccharomyces Genome Database

(S288C_reference_sequence_R64-3-1_20210421). Also added to this file was the genome for T4 phage (OL964735.1; GenBank) as this sequence appears in some sequencing datasets due to use in other ongoing experiments. Finally, for some samples, reads that did not map to any of the listed genomes were assembled using SPADES version 3.13.0 with default parameters. When these contigs were input into Blastn it revealed the presence of the aforementioned T4 phage DNA (subsequently added to reference file) but also plasmids of S. cerevisiae, and S. enterica that were not included in the reference genomes (S. cerevisiae: CP059538.1, J01347.1; S. enterica: CP012345.2; GenBank). These plasmids were also added to the reference genome file.

In silico digest of C. elegans genome. The C. elegans genome was digested in silico based on sites where a Dam or Dcm cleavage site is expected. Each read mapped by Bowtie2 was located to the theoretical fragment by genomic coordinates. The theoretical length of the containing fragment(s) for each read was assessed by measuring the number of bases between upstream and downstream cut sites.

Data Availability. All sequencing datasets used in this study have been deposited on to the NCBI SRA (PRJNA903933). SRA accession numbers for each sample can be found in the supplementary excel file.

### Example 2

Methylation-Dependent Enrichment of E. coli and Enterobacterial DNA

This procedure uses the differential presence of Dam+Dcm methylation in enterobacteria and other organisms in a metagenomic sample to enrich for enterobacterial DNA for downstream sequencing. Most enterobacteria including E. coli and S. enterica methylate almost all of GATC (Dam) and CCWGG (Dcm) sites. There are restriction endonucleases, namely PspGI, Mbol and EcoRII that will cut only unmethylated versions of these sites leaving enterobacterial sequences intact but degrading other sequences. T5 exonuclease can then be used to eliminate short fragments from the endonuclease treatment so that mostly longer enterobacterial sequences remain in the sample and can be sequenced. The distributive nature of T5 exonuclease treatment can be substantially advantageous in the protocol; highly processive nucleases that act sequentially to degrade DNA molecules can be much less appropriate for the consistent degradation of shorter DNA, particularly in cases where the individual rate of degradation for individual DNAs once targeted from the end is very rapid.

### Materials

Endonuclease reaction: PspGI (NEB R0611S), Mbol (NEB R0147S), EcoRII (TFS ER1921), rCutSmart™ Buffer (NEB B6004SVIAL), 2M NaCl, 6× NEB Purple Loading Dye (B7024S) supplemented with an extra 6 mM of EDTA to make the 1X solution 11 mM EDTA 4 (NEB

Exonuclease reaction: T5 exonuclease (NEB M0663S), NEBuffer™ 4 B7004SVIAL), 6X NEB Purple Loading Dye (B7024S) supplemented with an extra 6 mM of EDTA to make the 1X solution 11 mM EDTA.

Clean and Concentrate DNA: Genomic DNA Clean & Concentrator-10 (Zymo D4011), 10 mM Tris-CI pH 8.5.

### Procedure

Endonuclease reaction. Add the following reagents to a 1.5 mL reaction tube on ice (volumes given for a 25 μL reaction): 0.5 ug DNA, rCutSmart Buffer to 1× (2.5 μL of 10x), 4U of PspGI (0.4 μL of 10,000 units/mL) (add at end), Water to 25 μL, Mix by pipetting and incubate at 50C for 30 mins, Add 2U (0.4 μL) of Mbol, mix by pipetting and incubate at 37 C for 30 mins. Add the following reagents to the reaction tube: Supplement the buffer with NaCl to a final concentration of 50 mM (0.625 μL of 2M NaCl) Note: EcoRII as obtained from Thermo Fisher Scientific has been optimized in a different buffer (O buffer) and the addition of NaCl to the rCutSmart buffer is to bring salt conditions close to that of the O buffer. 4U of EcoRII (0.4 μL). Mix by pipetting and incubate at 37 C for 1 hour.

(Optional) The reaction may be stopped with 11 mM EDTA and then run through a Zymo Clean and Concentrate kit before exonuclease treatment.

Exonuclease reaction: Add 2.7U (0.27 μL) of T5 Exonuclease to the sample. Incubate at 37C for 20 mins. Immediately add 6X NEB Purple Dye supplemented with an extra 6 mM of EDTA to a concentration of 1X (5.33 μL). Note: 1× NEB Purple Dye contains 10 mM EDTA and supplementing it to 11 mM EDTA will put it in excess of the magnesium in the buffer to stop the reaction.

Clean and Concentrate DNA: Purified using components from Zymo Research (Zymo Genomic DNA Clean & Concentrator-10 kit D4011) Eluted with 10 mM Tris-CI pH8.5 buffer (12 μL).

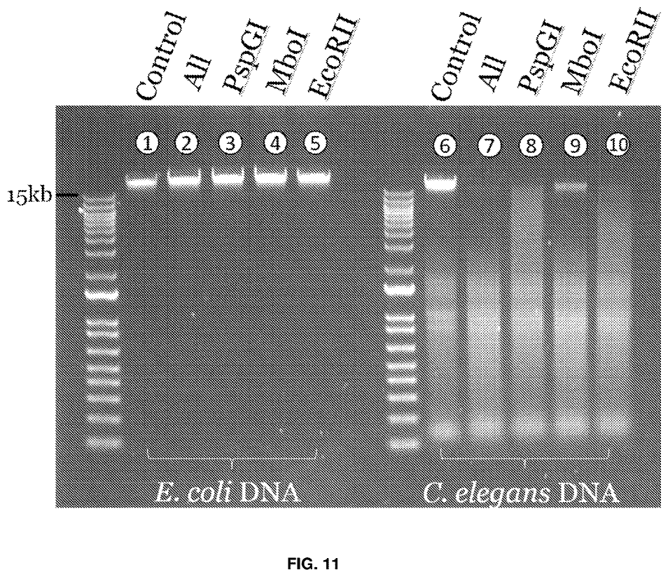

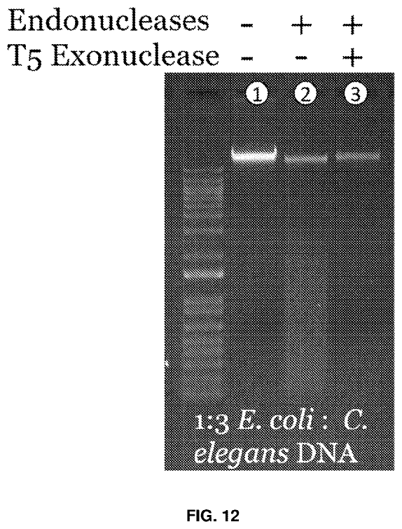

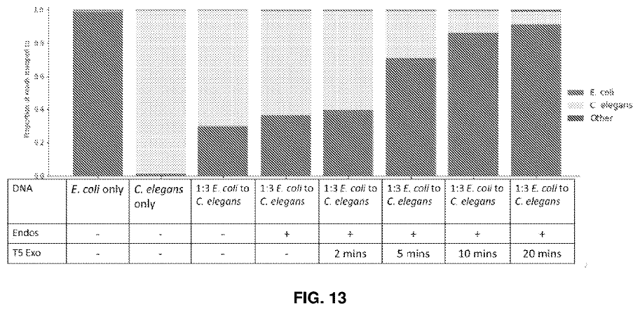

Results of a sample endonuclease and exonuclease digestion are shown in FIGS. 11 and 12. Assay for purification by sequencing is shown in FIG. 13.

### REFERENCES

1. Buytaers F E, Saltykova A, Denayer S, Verhaegen B, Vanneste K, Roosens N H C, Pierard D, Marchal K, De Keersmaecker S C J. 2020. A Practical Method to Implement Strain-Level Metagenomics-Based Foodborne Outbreak Investigation and Source Tracking in Routine. Microorganisms 8: E1191.
2. Deng X, den Bakker H C, Hendriksen R S. 2016. Genomic Epidemiology: Whole-Genome-Sequencing-Powered Surveillance and Outbreak Investigation of Foodborne Bacterial Pathogens. Annual Review of Food Science and Technology 7:353-374.
3. Buytaers F E, Saltykova A, Mattheus W, Verhaegen B, Roosens N H C, Vanneste K, Laisnez V, Hammami N, Pochet B, Cantaert V, Marchal K, Denayer S, De Keersmaecker S C J. 2021. Application of a strain-level shotgun metagenomics approach on food samples: resolution of the source of a Salmonella food-borne outbreak. Microb Genom 7:000547.
4. Saltykova A, Buytaers F E, Denayer S, Verhaegen B, Pierard D, Roosens N H C, Marchal K, De Keersmaecker S C J. 2020. Strain-Level Metagenomic Data Analysis of Enriched In Vitro and In Silico Spiked Food Samples: Paving the Way towards a Culture-Free Foodborne Outbreak Investigation Using STEC as a Case Study. Int J Mol Sci 21: E5688.
5. Buytaers F E, Saltykova A, Denayer S, Verhaegen B, Vanneste K, Roosens N H C, Pierard D, Marchal K, De Keersmaecker S C J. 2021. Towards Real-Time and Affordable Strain-Level Metagenomics-Based Foodborne Outbreak Investigations Using Oxford Nanopore Sequencing Technologies. Frontiers in Microbiology 12.
6. Forghani F, Li S, Zhang S, Mann D A, Deng X, den Bakker H C, Diez-Gonzalez F. 2020. Salmonella enterica and Escherichia coli in Wheat Flour: Detection and Serotyping by a Quasimetagenomic Approach Assisted by Magnetic Capture, Multiple-Displacement Amplification, and Real-Time Sequencing. Applied and Environmental Microbiology 86: e00097-20.
7. Fratamico P M, DebRoy C, Needleman D S. 2016. Editorial: Emerging Approaches for Typing, Detection, Characterization, and Traceback of Escherichia coli. Frontiers in Microbiology 7.
8. Barrangou R, Dudley E G. 2016. CRISPR-Based Typing and Next-Generation Tracking Technologies. Annual Review of Food Science and Technology 7:395-411.
9. Deng X, Shariat N, Driebe E M, Roe C C, Tolar B, Trees E, Keim P, Zhang W, Dudley E G, Fields P I, Engelthaler D M. 2015. Comparative Analysis of Subtyping Methods against a Whole-Genome-Sequencing Standard for Salmonella enterica Serotype Enteritidis. Journal of Clinical Microbiology 53:212-218.
10. Franz E, Gras L M, Dallman T. 2016. Significance of whole genome sequencing for surveillance, source attribution and microbial risk assessment of foodborne pathogens. Current Opinion in Food Science 8:74-79.
11. Barnes H E, Liu G, Weston C Q, King P, Pham L K, Waltz S, Helzer K T, Day L, Sphar D, Yamamoto R T, Forsyth R A. 2014. Selective Microbial Genomic DNA Isolation Using Restriction Endonucleases. PLOS One 9: e109061.
12. Liu G, Weston C Q, Pham L K, Waltz S, Barnes H, King P, Sphar D, Yamamoto R T, Forsyth RA. 2016. Epigenetic Segregation of Microbial Genomes from Complex Samples Using Restriction Endonucleases Hpall and McrB. PLOS ONE 11: e0146064.
13. Chiou K L, Bergey C M. 2018. Methylation-based enrichment facilitates low-cost, noninvasive genomic scale sequencing of populations from feces. 1. Sci Rep 8:1975.
14. Marotz C A, Sanders J G, Zuniga C, Zaramela L S, Knight R, Zengler K. 2018. Improving saliva shotgun metagenomics by chemical host DNA depletion. Microbiome 6:42.
15. Heravi F S, Zakrzewski M, Vickery K, Hu H. 2020. Host DNA depletion efficiency of microbiome DNA enrichment methods in infected tissue samples. Journal of Microbiological Methods 170:105856.
16. Feehery G R, Yigit E, Oyola S O, Langhorst B W, Schmidt V T, Stewart F J, Dimalanta E T, Amaral-Zettler L A, Davis T, Quail M A, Pradhan S. 2013. A Method for Selectively Enriching Microbial DNA from Contaminating Vertebrate Host DNA. PLOS ONE 8: e76096.
17. Takahashi Y, Shoura M, Fire A, Morishita S. 2022. Context-dependent DNA polymerization effects can masquerade as DNA modification signals. BMC Genomics 23:249.
18. O'Brown Z K, Boulias K, Wang J, Wang S Y, O'Brown N M, Hao Z, Shibuya H, Fady P-E, Shi Y, He C, Megason S G, Liu T, Greer EL. 2019. Sources of artifact in measurements of 6 mA and 4mC abundance in eukaryotic genomic DNA. BMC Genomics 20:445.
19. Oliveira P H, Fang G. 2021. Conserved DNA Methyltransferases: A Window into Fundamental Mechanisms of Epigenetic Regulation in Bacteria. Trends Microbiol 29:28-40.
20. Wion D, Casadesus J. 2006. N6-methyl-adenine: an epigenetic signal for DNA-protein interactions. 3. Nat Rev Microbiol 4:183-192.
21. Mouammine A, Collier J. 2018. The impact of DNA methylation in Alphaproteobacteria. Molecular Microbiology 110:1-10.
22. Løbner-Olesen A, Skovgaard O, Marinus MG. 2005. Dam methylation: coordinating cellular processes. Current Opinion in Microbiology 8:154-160.
23. Marinus M G, Morris N R. 1973. Isolation of deoxyribonucleic acid methylase mutants of Escherichia coli K-12. J Bacteriol 114:1143-1150.
24. Geier G E, Modrich P. 1979. Recognition sequence of the dam methylase of Escherichia coli K12 and mode of cleavage of Dpn I endonuclease. J Biol Chem 254:1408-1413.
25. Marinus M G, Løbner-Olesen A. 2014. DNA Methylation. EcoSal Plus 6:10.1128/ecosalplus.ESP-0003-2013.
26. Cornish-Bowden A. 1985. Nomenclature for incompletely specified bases in nucleic acid sequences: recommendations 1984. Nucleic Acids Res 13:3021-3030.
27. May M S, Hattman S. 1975. Analysis of bacteriophage deoxyribonucleic acid sequences methylated by host- and R-factor-controlled enzymes. J Bacteriol 123:768-770.
28. Palmer B R, Marinus M G. 1994. The dam and dom strains of Escherichia coli-a review. Gene 143:1-12.
29. Joannes M, Saucier J M, Jacquemin-Sablon A. 1985. DNA filter retention assay for exonuclease activities. Application to the analysis of processivity of phage T5 induced 5′-exonuclease. Biochemistry 24:8043-8049.
30. Sato M P, Ogura Y, Nakamura K, Nishida R, Gotoh Y, Hayashi M, Hisatsune J, Sugai M, Takehiko I, Hayashi T. 2019. Comparison of the sequencing bias of currently available library preparation kits for Illumina sequencing of bacterial genomes and metagenomes. DNA Research 26:391-398.
31. Schwartz D C, Cantor C R. 1984. Separation of yeast chromosome-sized DNAs by pulsed field gradient gel electrophoresis. Cell 37:67-75.
32. Bankevich A, Nurk S, Antipov D, Gurevich A A, Dvorkin M, Kulikov A S, Lesin V M, Nikolenko S I, Pham S, Prjibelski A D, Pyshkin A V, Sirotkin A V, Vyahhi N, Tesler G, Alekseyev M A, Pevzner P A. 2012. SPAdes: A New Genome Assembly Algorithm and Its Applications to Single-Cell Sequencing. J Comput Biol 19:455-477.
33. Josse J, Kornberg A. 1962. Glucosylation of Deoxyribonucleic Acid: III. α-AND β-GLUCOSYL TRANSFERASES FROM T4-INFECTED ESCHERICHIA COLI. Journal of Biological Chemistry 237:1968-1976.
34. Pratt E A, Kuno S, Lehman I R. 1963. Glucosylation of the deoxyribonucleic acid in hybrids of coliphages T2 and T4. Biochimica et Biophysica Acta (BBA)—Specialized Section on Nucleic Acids and Related Subjects 68:108-111.
35. Flodman et al. 2020. In vitro Type II Restriction of Bacteriophage DNA With Modified Pyrimidines. Frontiers in Microbiology 11.
36. Pightling A W, Petronella N, Pagotto F. 2014. Choice of reference sequence and assembler for alignment of Listeria monocytogenes short-read sequence data greatly influences rates of error in SNP analyses. PLOS One 9: e104579.
37. Militello K T, Simon R D, Qureshi M, Maines R, Van Horne M L, Hennick S M, Jayakar S K, Pounder S. 2012. Conservation of Dcm-mediated cytosine DNA methylation in Escherichia coli. FEMS Microbiology Letters 328:78-85.
38. Gomez-Eichelmann M C, Levy-Mustri A, Ramirez-Santos J. 1991. Presence of 5-methylcytosine in CC (A/T) GG sequences (Dcm methylation) in DNAs from different bacteria. Journal of Bacteriology 173:7692-7694.
39. 2009. The genome sequence of E. coli OP50. The WBG. Retrieved 6 Sep. 2022.
40. On Y Y, Welch M 2021. The methylation-independent mismatch repair machinery in Pseudomonas aeruginosa. Microbiology 167:001120.
41. Fang et al. 2012. Genome-wide mapping of methylated adenine residues in pathogenic Escherichia coli using single-molecule real-time sequencing. 12. Nat Biotechnol 30:1232-1239.
42. Sanjar F, Hazen T H, Shah S M, Koenig S S K, Agrawal S, Daugherty S, Sadzewicz L, Tallon L J, Mammel M K, Feng P, Soderlund R, Tarr P I, DebRoy C, Dudley E G, Cebula T A, Ravel J, Fraser C M, Rasko D A, Eppinger M. 2014. Genome Sequence of Escherichia coli 0157: H7 Strain 2886-75, Associated with the First Reported Case of Human Infection in the United States. Genome Announcements 2: e01120-13.
43. Jeudy S, Rigou S, Alempic J-M, Claverie J-M, Abergel C, Legendre M. 2020. The DNA methylation landscape of giant viruses. 1. Nat Commun 11:2657.
44. Yang Y, Feye K M, Shi Z, Pavlidis H O, Kogut M, J Ashworth A, Ricke S C. 2019. A Historical Review on Antibiotic Resistance of Foodborne Campylobacter. Front Microbiol 10:1509.
45. Kollef M H, Torres A, Shorr A F, Martin-Loeches I, Micek S T. 2021. Nosocomial Infection. Critical Care Medicine 49:169-187.
46. Howard A, O'Donoghue M, Feeney A, Sleator R D. 2012. Acinetobacter baumannii. Virulence 3:243-250.
47. Podschun R, Ullmann U. 1998. Klebsiella spp. as Nosocomial Pathogens: Epidemiology, Taxonomy, Typing Methods, and Pathogenicity Factors. Clin Microbiol Rev 11:589-603.
48. Nicholas R A J, Ayling R D. 2003. Mycoplasma bovis: disease, diagnosis, and control. Research in Veterinary Science 74:105-112.
49. Cao et al. 2022. mEnrich-seq: Methylation-guided enrichment sequencing of bacterial taxa of interest from microbiome. bioRxiv https://doi.org/10.1101/2022.11.07.515285.
50. Szekeres M, Matveyev A V. 1987. Cleavage and sequence recognition of 2,6-diaminopurine-containing DNA by site-specific endonucleases. FEBS Letters 222:89-94.
51. Green M R, Sambrook J. 2017. Isolating DNA from Gram-Negative Bacteria. Cold Spring Harb Protoc 2017: pdb.prot093369.
52. Langmead B, Salzberg SL. 2012. Fast gapped-read alignment with Bowtie 2. Nat Methods 9:357-359.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

The invention claimed is:

1. A method to enrich for DNA corresponding to a genome of interest from a mixed DNA population, the method comprising:
digesting the mixed DNA population with at least one selective endonuclease blocked by or enabled by a DNA modification, where the sample is suspected of containing a portion of DNA that comprises the modification to generate an endonuclease digested DNA population; and
retaining preferentially the undigested DNA after endonuclease treatment, wherein DNA corresponding to the genome of interest is enriched.

2. The method of claim 1, wherein the step of retaining preferentially the undigested DNA after endonuclease treatment comprises size selection by electrophoretic separation, and extraction of selected DNA fragments.

3. The method of claim 1, wherein the step of retaining preferentially the undigested DNA after endonuclease treatment comprises digesting the endonuclease-treated population with an exonuclease for a period of time sufficient to degrade endonuclease-cleaved DNA.

4. The method of claim 3, where the exonuclease acts distributively on the population of endonuclease-cleaved DNA molecules.

5. The method of claim 3, comprising selecting the exonuclease cleaved DNA by size for fragments of greater than about 5 kilobases in length.

6. The method of claim 1, further comprising generating a library from the preferentially retained DNA.

7. The method of claim 1, further comprising sequencing the preferentially retained DNA.

8. The method of claim 1, wherein the mixed DNA population comprises a mixture of prokaryotic and eukaryotic DNA, wherein all or a portion of the prokaryotic DNA is preferentially retained, optionally wherein the retained DNA is selected Enterobacteriaceae DNA, optionally E. coli, Salmonella, or Shigella DNA.

9-10. (canceled)

11. The method of claim 1, wherein the nucleic acid sample is a food sample, an environmental sample, or a clinical sample.

12-13. (canceled)

14. The method of claim 1, wherein the DNA modification is methylation.

15. The method of claim 14, wherein the methylation is one or both of Dam methylation and Dcm methylation.

16. The method of claim 1, wherein the at least one endonuclease is a restriction endonuclease.

17. The method of claim 16, wherein the at least one restriction endonuclease is selected from PspGI, EcoRII, and Mbol.

18. The method of claim 17, wherein the at least one restriction endonuclease is a cocktail of PspGI, EcoRII, and Mbol.

19. The method of claim 3, wherein the exonuclease is T5 nuclease.

20. A method for characterizing enterobacterial DNA in a mixed DNA sample, the method comprising:
obtaining a nucleic acid sample of interest, comprising a mixture of microbial and possibly non-microbial samples;
treating the nucleic acid sample with a cocktail of enzymes specific for one or at least two different recognition sites, where the enzymes are blocked or enabled by methylation at said recognition sites;
treating the endonuclease digested DNA with a DNA exonuclease for a period of time sufficient to selectively eliminate shorter, endonuclease cleaved fragments; and
characterizing the origin of remaining DNA segments.

21. The method of claim 20, wherein the microbial DNA includes DNA suspected of being from a microbial pathogen or pathogens.

22. The method of claim 21, wherein the suspected microbial pathogen or pathogens include pathogenic Enterobacteriaceae.

23. The method of claim 20, wherein the sample is a food sample, an environmental sample, or a clinical sample.

24. A kit for use in the methods of claim 1.
